(19) United States
(12) Patent Application Publication
(10) Pub. No.: US 2015/0223675 A1
(43) Pub. Date: Aug. 13, 2015

(54) LUMINAL ADMINISTRATION OF TAG MOLECULES FOR DIAGNOSTIC APPLICATIONS

(71) Applicant: GIVEN IMAGING LTD., Yoqneam (IL)

(72) Inventors: Osnat Sella-Tavor, Kfar Kish (IL); Elisha Rabinovitz, Haifa (IL)

(73) Assignee: GIVEN IMAGING LTD., Yoqneam (IL)

(21) Appl. No.: 14/425,062

(22) PCT Filed: Sep. 3, 2013
(86) PCT No.: PCT/IL13/50747

§ 371 Date: Mar. 2, 2015

Related U.S. Application Data
(60) Provisional application No. 61/696,380, filed on Sep. 4, 2012.
(60) Provisional application No. 61/756,697, filed on Jan. 25, 2013.

(51) Int. Cl. A61B 1/04; A61M 31/00; A61K 49/00; A61B 1/31; A61B 1/00; A61B 5/00; A61B 1/273

(52) U.S. Cl. CPC A61B 1/043; A61B 5/0075; A61M 31/005; A61B 5/0071; A61B 5/7282; A61B 5/4238; A61B 5/4255; A61B 1/041; A61B 1/2736; A61B 1/31; A61B 1/00009; A61K 49/0034

(57) ABSTRACT

Methods of tagging cancerous tissue for diagnostic applications, mainly for cancer screening purposes, include oral or luminal administrating of a tag molecule into a patient's gastrointestinal lumen either orally or through an enema, and tagging cancerous tissue present within the lumen with the tag molecule. The tag molecule may be a near infrared dye, e.g., IR783 or a derivative thereof, which showed to be of high concentration within cancerous tissue compared to normal tissue. The near infrared IR783 dye is administered, either orally or through an enema, as part of a pharmaceutical composition or formulation either as is or conjugated to a specific polymer. The formulation may comprise an enteric coating in order to keep the formulation intact prior to it reaching the target organ, which is to be tagged by the formulation.

### FIELD OF THE INVENTION

The invention relates to formulations and methods of luminal administration of tag molecules for diagnostic applications.

### BACKGROUND OF THE INVENTION

It is well known that early diagnosis of cancerous tissue is crucial for increasing the chances of a patient to overcome his disease. Therefore, periodical screening of the population for various types of cancers is important. Nowadays, people above the age of 50 are encouraged by their physician to undergo endoscopy procedures every couple of years, in order to ensure that they do not suffer from colon cancer. However, although people are aware of the importance of early detection of cancer, most of them are reluctant to undergo such invasive and somewhat uncomfortable procedures.

Colonic polyps are slow-growing overgrowths of the colonic mucosa, highly prevalent in the general population, especially with increasing age. While approximately 90% of the polyps are less than 1 cm in diameter and have a low potential for malignancy, the remaining 10% of adenomatous polyps especially those that are larger than 1 cm in diameter, and those of which containing a substantial (>25%) villous component or having high-grade dysplasia commonly carry an increased cancer risk.

Efficient marking of those adenomas by optical probes may significantly improve the detection of cancer.

Some methods of early screening are available. For example, swallowing an autonomous capsule endoscope imaging device, such as the PillCam®, produced by Given Imaging Ltd., Yoqneam, Israel. The capsule endoscope moves along the gastrointestinal tract by natural peristalsis, acquiring images of the lumen as it passes through it. However, in some cases, as may well happen when using standard endoscopes, it may be difficult to identify pathological areas along the lumen, as pathologies may not stand out much compared to the lumen's background.

Therefore, methods of “highlighting” the pathologies compared to the lumen's background have developed. For example, dying the cancerous and/or the adenoma tissue lumen with near infrared color may assist in easily distinguishing between the cancerous tissue and the non-cancerous tissue surrounding it. Near infrared imaging of internal tissues is typically being used, since near infrared radiation exhibits penetration of up to 8 centimeters into the irradiated tissue. This may provide information concerning tumors and polyps that are exposed to the lumen but do not necessarily protrude much out of the lumen wall.

International Patent Application Publication Number W02011086548 discloses using a conjugate of a specific polymer and a near infrared dye such as Cy5, Cy5.5 Indocyanine green (ICG), IR783, and analogs thereof. The polymer with the near infrared dye is shown to be inserted into diseased tissue by various means.

“Near IR Heptamethine Cyanine Dye-Mediate Cancer Imaging”, 2010, American Association for Cancer Research, by Xiaojian Yang et al., and “A near-infrared fluorescent heptamethine indocyanine dye with preferential tumor accumulation for in vivo imaging”, Biomaterials 31 (2010), by Chao Zhang et al., disclose the accumulation of near infrared dye IR783 in human cancer cells, and the dye's retention in those cells and not in normal cells. The human cancer cells were implanted into nude mice, and the near infrared dye was injected into the mice.

Therefore, for improved compliance by healthy population, specific distribution in the gastrointestinal system and less expected adverse effects, there is a need for methods and formulations for luminal administration of the infrared dye for in-vivo detection of cancer in the gastrointestinal system, such as, for example, colon cancer, adenomatous colorectal polyps, stomach cancer or small bowel cancer.

### SUMMARY OF THE INVENTION

The present invention describes methods of luminal administration of tag molecules for diagnostic applications. For purposes of screening the population for detection of cancer (e.g., colon cancer, stomach cancer and/or small bowel cancer), there is a need for a simple and user-friendly method of in-vivo insertion of the near infrared dyes other than injection of the dye. Therefore, the present invention discloses administering the near infrared dye, which may be, for example, IR-783, by luminal administration, i.e., either through oral administration by swallowing a formulation comprising the dye, or through an enema. These two administration methods are simple and comfortable for the patient to undergo and would increase patient compliance to cancer screening methods.

In some embodiments of the present invention, the near infrared dye (e.g., IR-783) may be part of a delivery composition or formulation that comprises a coating (e.g., an enteric coating) or other means of keeping the dye intact prior to reaching the target organ, which is to be tagged. Such delivery compositions are specifically required when the near infrared dye is to be inserted orally.

According to embodiments of the invention, a method of tagging cancerous tissue may comprise the steps of administrating a tag molecule into a patient's gastrointestinal lumen either orally or through an enema, and tagging cancerous tissue present within the lumen with the tag molecule. According to some embodiments, the tag molecule may be a near infrared dye, for example, without limitation, IR-783.

According to some embodiments, the method may further comprise the step of imaging the tagged cancerous tissue. In some embodiments, the step of imaging the tagged cancerous tissue may be conducted by a swallowable imaging capsule. In other embodiments, the step of imaging the tagged cancerous tissue may be conducted by an endoscope.

In some embodiments, the cancerous tissue may be selected from a group consisting of colon cancer, stomach cancer, small bowel cancer, or a combination thereof.

In some embodiments, the invention provides an enteric coated formulation comprising a near infrared dye and a pharmaceutically acceptable carrier. In some embodiments, the coating of the enteric coated formulation comprises methacrylic acid copolymer, which may be Eudragit®L, Eudragit® S, Eudragit® RS, Eudragit® RL, Eudragit® FS 30P, and Eudragit® NE, or a cellulose derivative or any combination thereof.

In some embodiments, the near infrared dye is a fluorescent probe which is IR-783, a derivative of IR-783, e.g., IR-783-S-Ph-COOH, IR-780 iodide, IR-786 iodide, rhodamine 123, a dye such as indocyanine green (ICG), Cy5, Cy5.5, Cy5.18, Cy7 and Cy7.18, IRDye 78, IRDye 680, IRDye 750, IRDye 800 phosphoramidite, DY-681 , DY-731 , DY-781 Alexa Fluor 610, Alexa Fluor 633 , Alexa Fluor 647, Alexa Fluor 660, Alexa Fluor 680, Alexa Fluor 700 and Alexa Fluor 750.

In some embodiments, the formulation may be in the form of a tablet or a capsule. In some embodiments, there is provided a formulation comprising a near infrared dye in a form for being administered in an enema.

In some embodiments of the invention, there is provided a method for detecting or imaging adenoma and/or cancer or pre-cancerous cells in a gastrointestinal system of a subject. The method may comprise the steps of administering to said subject, either orally or through an enema, an effective amount of a formulation comprising a near infrared dye as defined above, and detecting or imaging cells in the gastrointestinal tract lumen that take up said near infrared dye, to determine if cancer is present in the gastrointestinal system of the subject.

In some embodiments, the method further includes a step of removing unbound or unspecifically bound near infrared dye.

According to some embodiments, the cancer to be detected is colon cancer, adenomatous colorectal polyps, stomach cancer or small bowel cancer. In some embodiments, the detecting step is conducted by an endoscope or by swallowing an autonomous capsule endoscope imaging device. In some embodiments, the autonomous capsule endoscope imaging device is the PillCam®.

According to some embodiments, the method may comprise the following steps: administering to the patient, who is on a liquid diet at least a day prior to beginning of the procedure, either orally or through an enema, the formulation defined above, administering a laxative, administering the autonomous capsule endoscope imaging device, and administering a clear liquid agent.

### BRIEF DESCRIPTION OF THE DRAWINGS OF THE INVENTION

The principles and operation of the system and method according to the present invention may be better understood with reference to the drawings, and the following description, it being understood that these drawings are given for illustrative purposes only and are not meant to be limiting, wherein:

FIG. 1 is a graph illustrating the concentration of IR-783 in tumors compared to normal tissue, 24 hours after dye administration in whole body in vivo imaging , as disclosed in prior art.

FIG. 2 is a flow chart of a method of tagging cancerous tissue for diagnostic applications, according to an embodiment of the invention.

FIG. 3 is a flow chart of a method of tagging cancerous tissue for diagnostic applications, according to an embodiment of the invention;

FIG. 4 is a flow chart of a method for detecting or imaging cancer in a gastrointestinal system of a subject, according to an embodiment of the invention;

FIG. 5A shows a photograph of a colon from an orthotopic mouse with human colorectal tumors.

FIG. 5B shows a photograph of the mouse's colon after 4 μg of the IR-783 derivative (IR-783-S-PH-COOH) were introduced into the colon of the mouse via a minicolonoscope inserted through the mouse's rectum.

FIGS. 6A-B show photographs of colons of orthotopic mice with human colorectal tumors; FIG. 6A shows intensely labeled colon tumors, and FIG. 6B shows non-labeled colon tumors; and

FIG. 7 shows fluorescent photographs of the liver, spleen, heart, lungs, kidneys and brain of mice administered with 4 μg of IR-783 by gastric gavage.

### DETAILED DESCRIPTION OF THE INVENTION

In the following detailed description, numerous specific details are set forth in order to provide a thorough understanding of the invention. However, it will be understood by those of ordinary skill in the art that the present invention may be practiced without these specific details. In other instances, well-known methods, procedures, components and circuits have not been described in detail so as not to obscure the present invention.

The invention describes formulations and methods of luminal administration of tag molecules for diagnostic applications. For purposes of screening the population for detection of cancer (e.g., colon cancer, stomach cancer and/or small bowel cancer), there is a need for a simple and user-friendly method of in-vivo insertion of the near infrared dyes other than injection of the dye.

In one embodiment of the invention, there is provided an enteric coated formulation comprising a near infrared dye and a pharmaceutically acceptable carrier. The near infrared dye, which is also referred to herein as the tag molecule, may be a fluorescent probe which is IR-783, a derivative of IR-783, IR-780 iodide , IR-786 iodide, rhodamine 123, a dye such as indocyanine green (ICG), Cy5, Cy5.5, Cy5.18, Cy7 and Cy7.18, IRDye 78, IRDye 680, IRDye 750, IRDye 800 phosphoramidite, DY-681 , DY-731 , DY-781 Alexa Fluor 610, Alexa Fluor 633 , Alexa Fluor 647, Alexa Fluor 660, Alexa Fluor 680, Alexa Fluor 700 and Alexa Fluor 750, or any combination thereof.

In some embodiments of the invention, the near infrared dye is IR-783.

In some embodiments of the invention, the derivative of IR-783 is IR-783-S-PH-COOH.

According to some embodiments, the coating of the enteric coated formulation of the invention comprises methacrylic acid copolymer. The methacrylic acid copolymer may be one or more of Eudragit®L, Eudragit® S, Eudragit® RS, Eudragit® RL, Eudragit® FS 30P, and Eudragit® NE, or a cellulose derivative.

In some embodiments, there is provided a formulation comprising a near infrared dye in a form suitable for being administered in an enema. The near infrared dye may be a fluorescent probe which is IR-783, a derivative of IR-783, IR-780 iodide , IR-786 iodide, rhodamine 123, a dye such as indocyanine green (ICG), Cy5, Cy5.5, Cy5.18, Cy7 and Cy7.18, IRDye 78, IRDye 680, IRDye 750, IRDye 800 phosphoramidite, DY-681 , DY-731 , DY-781 Alexa Fluor 610, Alexa Fluor 633, Alexa Fluor 647, Alexa Fluor 660, Alexa Fluor 680, Alexa Fluor 700 and Alexa Fluor 750, or any combination thereof.

In some embodiments of the invention, the near infrared dye is IR-783.

The oral pharmaceutical compositions of the invention may also be formulated as a controlled-release matrix. For example, controlled-release matrix tablets in which the release of a soluble active ingredient is controlled by having the active ingredient diffuse through a gel formed after the swelling of a hydrophilic polymer brought into contact with a dissolving liquid (when used in vitro) or gastro-intestinal fluid (when used in vivo). Many polymers have been described as capable of forming such gel, e.g., derivatives of cellulose, cellulose ethers such as hydroxypropyl cellulose, hydroxymethyl cellulose, methylcellulose or hydroxypropyl methyl cellulose, or ethers having fairly high viscosity. According to other embodiments, the compositions comprise the active ingredient formulated for controlled release in a microencapsulated dosage form, in which small droplets of the active ingredient are surrounded by a coating or a membrane to form particles in the range of a few micrometers to a few millimeters.

Some embodiments for preparing the formulation are depot systems, which are based on biodegradable polymers, wherein as the polymer degrades the active ingredient is slowly released. Typically, hydrolytically labile polyesters prepared from lactic acid, glycolic acid, or combinations thereof may be used. Examples for biodegradable polymers prepared from these particular monomers include, without being limited to, poly(D,L-lactide) (PLA), polyglycolide (polyglycolic acid; PGA), and the copolymer poly(D,L-lactide-co-glycolide) (PLGA).

In some embodiment, the oral formulation may be in a form of a tablet. The tablet may comprise at least one filler, e.g., lactose, ethylcellulose, microcrystalline cellulose, silicified microcrystalline cellulose; at least one disintegrant, e.g., cross-linked polyvinylpyrrolidinone; at least one binder, e.g., polyvinylpyridone, hydroxypropylmethyl cellulose; at least one surfactant, e.g., sodium laurylsulfate; at least one glidant, e.g., colloidal silicon dioxide; and at least one lubricant, e.g., magnesium stearate.

In some embodiments, the oral formulation is in a form of a capsule.

In certain embodiments, the pharmaceutical composition or formulation of the invention, when formulated for oral administration, is in the form of a monolithic matrix, i.e., a structure including a three-dimensionally stable matrix material having a discrete size and shape; a tablet such as a bi-layered or multilayered tablet, matrix tablet, disintegrating tablet, dissolving tablet, or chewable tablet; or a capsule or sachet, e.g., filled with granules, grains, beads, or pellets.

In some embodiments of the invention, the amount of the near infrared dye is between 10 mg and 1000 mg. In some embodiments, the amount of the near infrared dye is between 10-200 mg. In some embodiments, the amount is about 10, 20, 30, 40, 50, 60, 70, 80, 90, 100, 110, 120, 130, 140, 150, 160, 170, 180, 190 or 200 mg±10%. It is noted that in some embodiments, the formulation may be administered to the patient more than one time prior to the detection procedure.

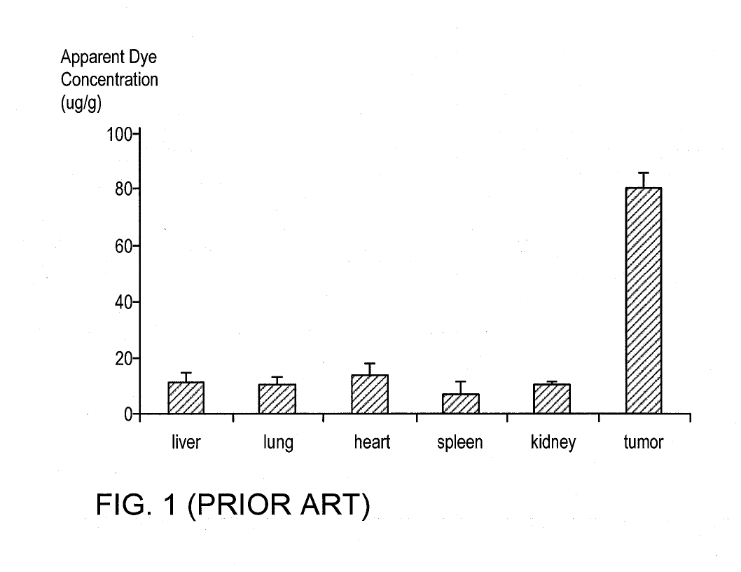

Reference is now made to FIG. 1, which is a graph illustrating the concentration of near infrared IR-783 dye in tumors compared to normal tissue, 24 hours after dye administration, by injection, in whole body in vivo imaging, as disclosed in the prior art. FIG. 1 shows that the IR-783 dye concentration is much higher in tumors compared to the dye concentration in normal body tissue. This proves that IR-783 may be efficiently used for tagging cancerous tissue and enabling sufficient differentiation between cancerous tissue and normal tissue surrounding it.

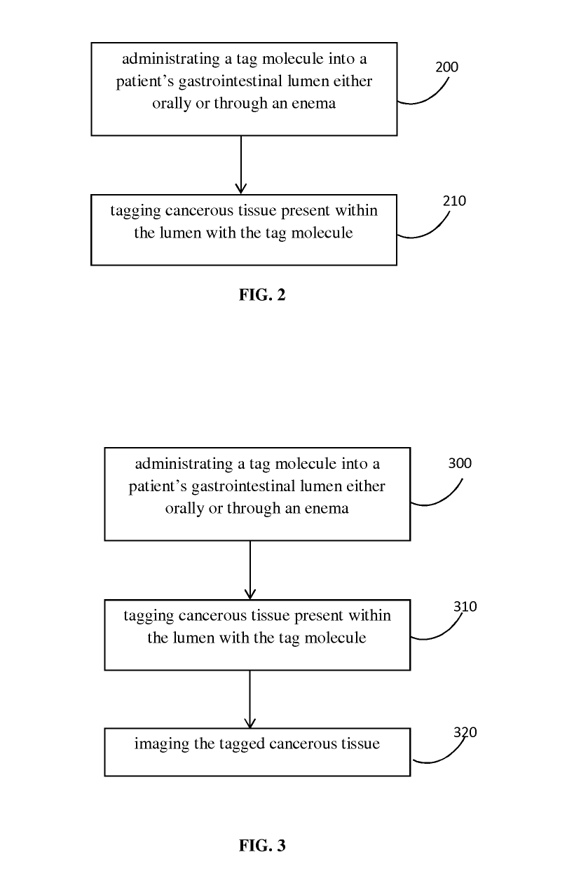

Reference is now made to FIG. 2, which is a flow chart of a method of tagging cancerous tissue for diagnostic applications, according to an embodiment of the invention. The method may comprise the following steps of administrating the formulation of the invention into a patient's gastrointestinal lumen either orally or through an enema (200), and tagging cancerous tissue present within the lumen with the tag molecule (210).

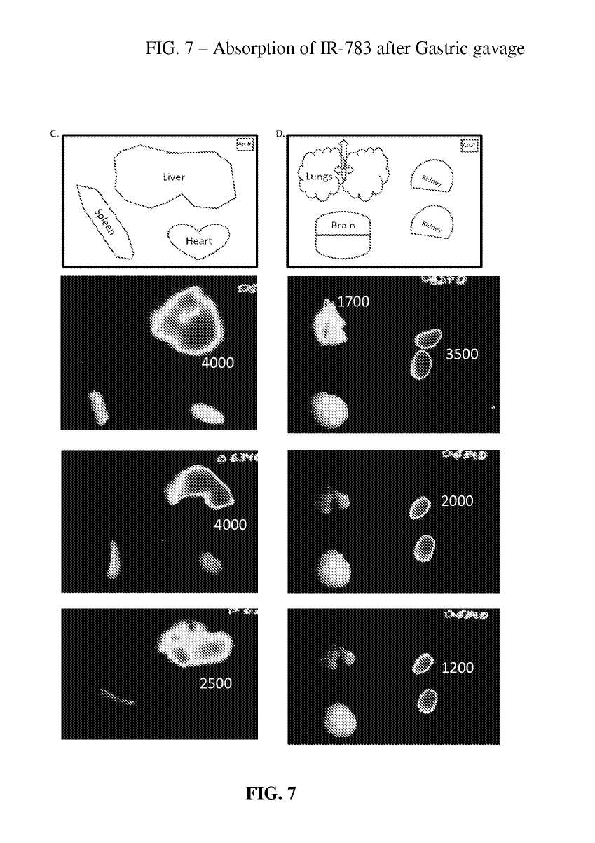

In some embodiments of the invention, the near infrared dye may be part of a delivery composition or formulation that comprises a coating or other means of keeping the dye intact prior to reaching the target organ. As explained in Example 3 and as shown in FIG. 7, part of the dye that was orally administered was absorbed in the stomach and thus did not reach the colon lumen. Therefore, formulations that protect the dye in the stomach for further release of it in the colon are required.

For example, the near infrared dye may be coated with enteric coating so that the dye may pass the acidic fluids of the stomach, while staying intact, until the dye reaches the small bowel or colon. Such delivery compositions are specifically required when the near infrared dye is to be inserted orally.

The step of tagging the cancerous tissue with the tag molecule may assist in diagnosing cancer within the lumen. The tissue tagged with a near infrared dye may be imaged with near infrared imaging devices, e.g., capsule endoscopes, standard endoscopes or other devices that incorporate near infrared imaging means. Since only the cancerous tissue is tagged with the tag molecule (e.g., IR-783), see Examples 1 and 2, it may be apparent from the images which section of the tissue is the cancerous tissue and which is normal tissue. Embodiments of the present invention may comprise using a swallowable in-vivo device/capsule, such as an autonomous swallowable capsule for imaging the tagged tissue. In other embodiments, the in-vivo device need not be swallowable or autonomous, and may have other shapes or configurations, e.g., a standard endoscope, though other imaging devices may be used.

According to some embodiments, the tissue that is to be tagged by the tag molecule (e.g., IR-783), may suffer from colon cancer, stomach cancer, small bowel cancer, or a combination thereof.

Reference is now made to FIG. 3, which is a flow chart of a method of tagging cancerous tissue for diagnostic applications, according to another embodiment of the invention. The method according to FIG. 3, may comprise the steps of: administrating the formulation of the invention into a patient's gastrointestinal lumen either orally or through an enema (300), tagging cancerous and or adenoma tissue present within the lumen with the tag molecule (310), and imaging the tagged cancerous tissue (320). The step of imaging the tagged cancerous tissue may be done by in-vivo devices such as capsule endoscopes, endoscopes or any other in-vivo device that may incorporate near infrared imaging means.

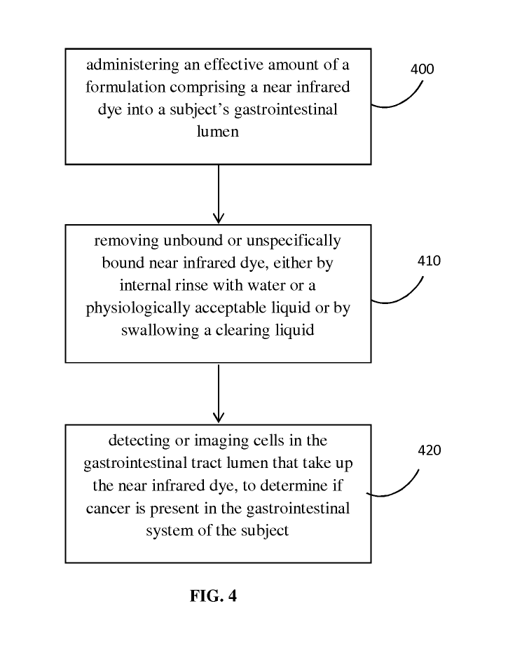

Reference is now made to FIG. 4, which is a flow chart of a method for detecting or imaging cancer in a gastrointestinal system of a subject, according to an embodiment of the invention. According to some embodiments, the method may comprise the steps of administering to said subject's gastrointestinal tract an effective amount of a formulation comprising a near infrared dye according to embodiments of the invention (400); and detecting or imaging cells in the gastrointestinal tract lumen that take up said near infrared dye, to determine if cancer is present in the gastrointestinal system of the subject (420). In some embodiments, the method may comprise a step of removing unbound or unspecifically bound near infrared dye, either by internal rinse with water or a physiologically acceptable liquid or by swallowing a clearing liquid (410). According to some embodiments of the invention, the invention provides a method for detecting or imaging pre-cancerous cells in a gastrointestinal system of a subject. The method may comprise the steps of: (a) administering to said subject an effective amount of a formulation comprising a near infrared dye according to an embodiment of the invention; and (b) detecting or imaging pre-cancerous cells that absorb or interact with the near infrared dye.

Prior to administration of the pharmaceutical composition, the gastrointestinal tract of the treated subject may be purged using appropriate fluids, and following administration, suitable fluids may further be administered to rinse non-attached particles so as to ensure that near infra-red (NIR) emission is detected essentially from regions having particles bound through their targeting moieties only, i.e., regions containing cancer or a pre-cancer tissues.

In some embodiments of the invention, the method involves a liquid diet for a day or more prior to beginning the procedure, i.e., before the step of administering the formulation of the invention. In some embodiments, a laxative is administered, before, after or simultaneously with the step of administering the formulation of the invention.

According to some embodiments of the invention, the method for detecting or imaging cancerous or pre-cancer cells in the gastrointestinal tract comprises administering to the patient, who is on a liquid diet for at least a day prior to the beginning of procedure, the formulation of the invention; administering a laxative, before, after or simultaneously with administering the formulation; and administering an autonomous capsule endoscope imaging device. In some embodiments, the method may further comprise the step of administering a clear liquid agent following the step of administering the autonomous capsule endoscope.

In some embodiments, for example, the patient may begin a liquid diet 24 hr prior to the procedure. On the evening of the first day, the patient is administered with the formulation of the invention, along with a laxative. In some embodiments, the laxative is administered before or after the formulation is administered. In the morning of the second day (i.e., the day of the procedure), the patient is administered with an autonomous capsule endoscope imaging device. In some embodiments of the invention, the patient may be administered with a booster laxative (to increase bowel activity), a few hours (e.g., between about 2 to 10 hr) after the administration of the autonomous capsule endoscope imaging device.

In some embodiments, near infrared emission is detected from the walls of the gastrointestinal tract by any suitable means, e.g., by means of colonoscopy or endoscopic capsules, a tube with an optical fiber, a swallowed capsule with a detector that transmits information to a receiver, or by an NIR detector that is placed outside the body.

In some embodiments of the invention, the methods of the present invention do not require processing of images; rather, in one embodiment, a surgeon or clinician, through the use of, e.g., intravascular probe or an endoscope, can quickly scan areas of suspected tumor growth and use the level of fluorescence to more precisely discriminate tumor tissue from non-tumor tissue and thereby more precisely define tumor borders for surgical resection or diagnostic evaluation, or for laser or radiation therapy, including brachytherapy and external beam therapy, or for improved biopsy procedures. In other embodiments, processing of images acquired by the imaging device, may either be performed by a processor external or internal to the imaging device.

In some embodiments of the invention, the method may be repeated one or more times so as to enable monitoring of the therapeutic effects of a medicament. The tumors in the patient's luminal tract may be detected by methods described hereinabove. The patient may then receive the appropriate medicament to treat the detected tumors. After a certain period of time, the patient's luminal tract may be monitored so as to assess whether the tumors' size was reduced or diminished.

In some embodiments of the invention there may be provided a kit with the oral formulation (or pharmaceutical composition) of the invention or with the formulation for an enema, or the combination thereof. In some embodiments, the kit may further comprise a laxative. In some embodiments, the kit may further comprise a clear liquid agent or a booster laxative. In some embodiments the kit may further comprise a laxative and a clear liquid agent or a booster laxative. In some embodiments, there may be provided a kit with the oral formulation of the invention or with the formulation for an enema, or the combination thereof, a laxative and a clear liquid agent (or a booster laxative) and instructions for the use and the regimen of each of the ingredients.

While certain features of the present invention have been illustrated and described herein, many modifications, substitutions, changes, and equivalents may occur to those of ordinary skill in the art. It is, therefore, to be understood that the appended claims are intended to cover all such modifications and changes as fall with the true spirit of the invention.

### EXAMPLES

### Example 1

Orthotopic mice with human colorectal tumors in their colons were used for gastric gavage administration of the IR-783 derivative—IR-783-S-PH-COOH. 4 μg of the IR-783-S-PH-COOH dissolved in 200 μl PBS were introduced into the colon of the mouse via minicolonoscope inserted through the rectum. The IR-783-S-PH-COOH solution was incubated in the colon for 20 minutes and then washed out with 5 ml PBS inserted through the rectum. Four hours later, mice were sacrificed and the fluorescence of the tumor and the surrounding colon tissue was measured by a fluorescent camera setup. Histopathological examination was performed on paraffin fixed colonic tissue specimens.

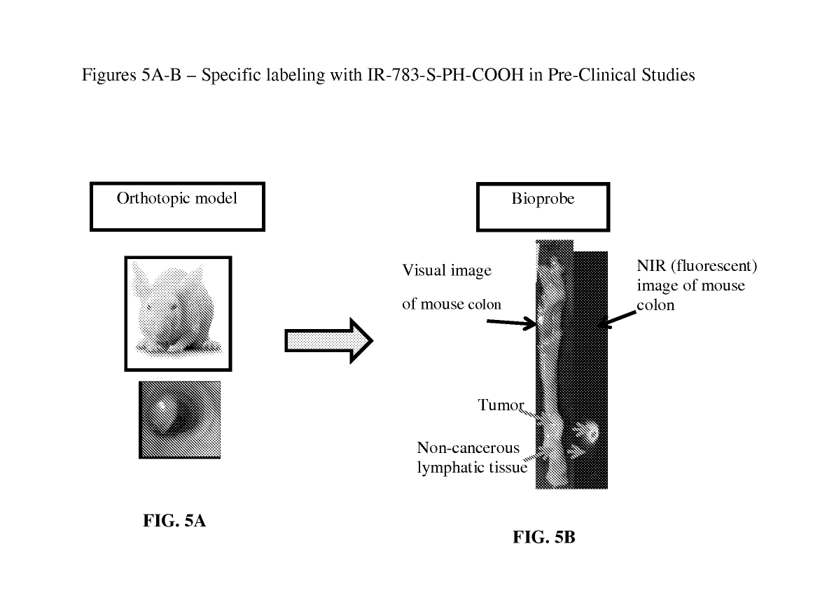

As can be seen from FIG. 5B, two regions suspected as tumors were observed. One of them was intensely labeled, with tumor to background ratio of 60 in 1 second exposure. This region was confirmed as a tumor in histopathologic examination. The second region was not labeled with the fluorescence material, and the histopatholgical examination confirmed that this region contains only non-cancerous lymphatic tissue with no tumor cells.

### Example 2

Orthotopic mice with human colorectal tumors in their colons were used for luminal administration of IR-783. 4 μg of IR-783 dissolved in 200 μl PBS were introduced into the colon of the mouse via a minicolonoscope inserted through the rectum. The IR-783 solution was incubated in the colon for 20 minutes and then washed out with 5 ml of PBS. 4 h later, the mice were sacrificed and the fluorescence of the tumor and the surrounding colon was measured by a fluorescent camera setup. Histopathological examination was performed on paraffin fixed colonic tissue specimens.

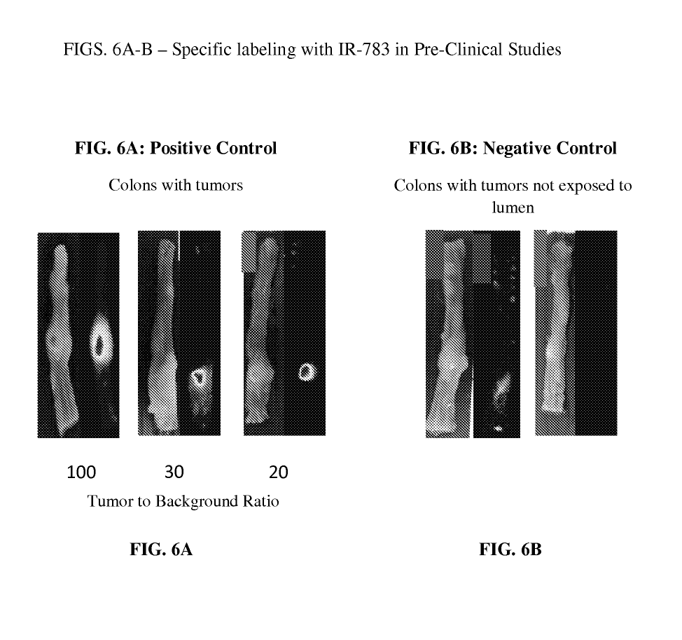

In FIG. 6A tumors intensely labeled are shown. Tumor to background ratios are indicated. Histopathologic examination of the labeled tumors confirmed the presence of tumor that grew over the murine mucosal layer and therefore are exposed to the mice colon lumen. In FIG. 6B tumors that were not labeled are shown. Further histopathology examination of these tumors verified that they were developed below the murine mucosal layer and therefore were not exposed to the colon lumen and could not become in contact with the IR-783 solution. Since colon cancer in humans is always developed over the mucosal layer (and not below the mucosal layer), it may be concluded that colon tumors in the human body may be efficiently and specifically labeled by IR783.

### Example 3

4 μg of IR-783 dissolved in 200 μl PBS were introduced into mice stomach by gastric gavage. Five hours later, the mice were sacrificed and liver, spleen, heart, lungs, kidneys and brain were collected and measured for fluorescence by a fluorescent camera setup. As can be seen from FIG. 7, fluorescence was detected in the liver and kidneys. Grey levels of the organs are indicated by the numbers on the fluorescent pictures next to the imaged organs, e.g., grey level in the liver was 4000 in two mice and 2500 in a third mouse, and grey levels in the kidneys were 3500, 200 and 1200, respectively.

These results suggest that part of the dye was absorbed in the stomach and thus did not reach the colon lumen. Therefore, formulations that protect the dye in the stomach for further release in the colon are required. For example, formulations with enteric coating may ensure the formulations stay intact prior to the formulations reaching the target location, e.g., the colon.

1-19. (canceled)

20. A method for detecting or imaging pre-cancerous cells and/or adenoma and/or cancer in a gastrointestinal system of a subject, said method comprising:
(a) providing the subject with an effective amount of a formulation for luminal administration, comprising a near infrared dye; and
(b) detecting or imaging cells that absorb or interact with said near infrared dye in the gastrointestinal system of said subject that received said formulation luminally, to determine if pre-cancer cells and/or cancer are present in the gastrointestinal system of the subject.

21. The method of claim 20, wherein the cancer is colon cancer, adenomatous colorectal polyps, stomach cancer or small bowel cancer.

22. The method of claim 20, wherein the method further comprises a step of removing unbound or unspecifically bound near infrared dye.

23. The method of claim 20, wherein the formulation is an enteric coated formulation comprising the near infrared dye and a pharmaceutically acceptable carrier.

24. The method of claim 23, wherein the coating of said enteric coated formulation comprises methacrylic acid copolymer.

25. The method of claim 24, wherein said methacrylic acid copolymer is Eudragit®L, Eudragit® S, Eudragit® RS, Eudragit® RL, Eudragit® FS 30P, and Eudragit® NE, or a cellulose derivative or any combination thereof.

26. The method of claim 20, wherein the near infrared dye is a fluorescent probe which is IR-783, a derivative of IR-783, IR-780 iodide, IR-786 iodide, rhodamine 123, a dye such as indocyanine green (ICG), Cy5, Cy5.5, Cy5.18, Cy7 and Cy7.18, IRDye 78, IRDye 680, IRDye 750, IRDye 800 phosphoramidite, DY-681 , DY-731 , DY-781 Alexa Fluor 610, Alexa Fluor 633, Alexa Fluor 647, Alexa Fluor 660, Alexa Fluor 680, Alexa Fluor 700 and Alexa Fluor 750.

27. The method of claim 20, wherein the near infrared dye is IR-783.

28. The method of claim 27, wherein the IR-783 derivative is IR-783-S-Ph-COOH.

29. The method of claim 20, wherein the formulation is in the form of a tablet, a capsule or an enema.

30. The method of claim 20, wherein the step of detecting is by an endoscope or by swallowing an autonomous capsule endoscope imaging device.

31. The method of claim 30, wherein the autonomous capsule endoscope imaging device is the PillCam®.

32. The method of claim 20, wherein at least a day prior to beginning of step (a) the subject is on a liquid diet.

33. The method of claim 32, further comprising a step of administering a laxative prior to step (b) and administering a clear liquid agent following step (b).
